(19) United States
(12) Patent Application Publication
(10) Pub. No.: US 2004/0106556 A1
(43) Pub. Date: Jun. 3, 2004

(54) Method of treating and preventing alzheimer disease through administration of delipidated protein and lipoprotein particles

(76) Inventors: Yanhong Zhu, Pleasanton, CA (US); Jo-Ann B. Maltais, San Ramon, CA (US); Bill E. Cham, Sheldon (AU)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/650,110

(22) Filed: Aug. 26, 2003

Related U.S. Application Data
(60) Provisional application No. 60/405,922, filed on Aug. 26, 2002.

(51) Int. Cl. A61K 38/17; A61K 31/56

(52) U.S. Cl. 514/12; 514/171

(57) ABSTRACT

The present invention relates to a method for preventing, reducing the progression of, and treating Alzheimer disease (AD), by creating delipidated protein particles and lipoprotein particles including at least one of LDL, VLDL, or HDL, other particles associated with lipid transport and metabolism, and combinations thereof, and administering these delipidated particles to patients at risk of developing AD or patients diagnosed with AD. The present invention employs energy and solvents useful for extracting lipids and cholesterol from the lipid-containing plasma and forming delipidated protein particles and lipoprotein particles. The present invention uses optimal solvent systems such that the lipid is dissolved while other plasma proteins are relatively intact. The present invention also provides a method of combining plasma delipidation and particle administration with other therapeutic interventions for prevention and treatment of AD.

### RELATED APPLICATIONS

This application claims the benefit of U.S. provisional patent application serial No. 60/405,922 filed Aug. 26, 2002.

### FIELD OF THE INVENTION

The present invention relates to methods of preventing, retarding progression of, and treating Alzheimer disease, involving removal of lipids and cholesterol from the plasma and administration of partially delipidated proteins and partially delipidated lipoproteins to patients at risk of developing or diagnosed with Alzheimer disease. The present method is optionally combined with other therapeutic approaches.

### BACKGROUND OF THE INVENTION

Alzheimer disease (AD) is a debilitating disease characterized by a loss of cognitive function associated with an excessive number of plaques in the cerebral cortex and subcortical gray matter, that contain beta amyloid (Aβ) and neurofibrillary tangles consisting of tau protein in its various forms. Patients having AD exhibit loss of memory, inability to learn and retain new information, language problems, mood swings, and difficulty performing tasks of daily living. The prevalence of AD approximately doubles with every five years over the age of 65, and currently there are over 12 million cases worldwide. It is expected that the number of Americans with AD will triple in the next 45 years to about 13 million. This crisis threatens the existence of the health care system and will intensify as longevity increases.

AD patients generally have a poor prognosis, with the average survival of a patient with AD being approximately 7 years. Currently there is no good method of treating AD patients. Some drugs may be used to temporarily improve memory during the early stages of the disease, but the drugs do not modify the steady progression of the disease. Many drugs simply increase confusion and lethargy. Therefore, an effective treatment for AD that will not contribute to the symptoms of the disease is needed.

AD is characterized by widespread neuronal degeneration, the presence of tau protein-rich intraneuronal neurofibrillary tangles, and the deposition of extracellular senile plaques whose major component is amyloid beta peptide (Aβ). Aβ included peptides of 40 (Aβ40) to 43 (Aβ43) amino acid residues in length. These Aβ peptides are derived from amyloid precursor protein (APP) through the sequential activity of β-secretase and γ-secretase. Increased Aβ formation leads to the elevated extracellular concentrations of Aβ42 or Aβ43. These peptides have a greater tendency to aggregate than Aβ40 and, therefore, are considered to be pathological. The increased release of Aβ42/Aβ43 leads to the abnormal deposition of Aβ and the associated neurotoxicity in the brains of affected individuals.

Epidemiologic data have shown that hypercholesterolemia is an important risk factor for AD. Some clinical studies have demonstrated a decreased prevalence of AD associated with the use of lipid-lowering drugs called statins to treat hypercholesterolemia. One study reported that statins reduced intracellular and extracellular levels of Ab42 and Ab40 peptides in primary cultures of hippocampal neurons and mixed cortical neurons. In another study, guinea pigs treated with statins exhibited a dramatic and reversible reduction of cerebral Aβ42 and Aβ40 levels in cerebrospinal fluid and the brain. These results suggest that cholesterol plays a role in the development of AD.

Accordingly, what is needed are new methods for treating, preventing or slowing the progression of AD through reduction of circulating cholesterol and lipid. What is also needed is a method which is simple, effective, and does not appreciably denature plasma proteins or extract them from the plasma.

### SUMMARY OF THE INVENTION

The present invention solves the problems described above by providing a simple, effective and efficient composition and method for decreasing circulating lipid and cholesterol in patients at risk of developing AD and patients suffering from AD. The present invention is effective in preventing AD, treating AD and slowing the progression of the disease. The therapeutic method of the present invention may be combined with other therapeutic approaches for reducing the risk of AD, reducing the progression of AD and treating AD.

The present invention comprises administration of one or more delipidated particles comprising high density lipoproteins (HDL), low density lipoproteins (LDL) or very low density lipoproteins (VLDL), or a combination thereof, to the patients at risk of developing or diagnosed with AD in an amount effective to prevent AD, to delay the progression of AD or to treat AD. These particles are prepared by chemically treating plasma containing these particles. Following preparation of these delipidated particles, they are administered to the patients at risk of developing or diagnosed with AD. Further, these particles are optionally combined with the patient's blood cells before administration to the patient.

The method of the present invention for creating these delipidated particles comprises removing lipid and cholesterol from these particles through a method comprising: obtaining blood containing the lipid, separating the blood cells from the plasma containing lipid, cholesterol and the protein particles and lipoprotein particles, contacting the plasma with a first organic solvent capable of solubilizing the lipid and cholesterol; and, separating a first phase containing the lipids and cholesterol from a second phase wherein the second phase is substantially free of the lipids and cholesterol. Particles in the delipidated plasma fraction may optionally be recombined with the blood cells and reintroduced into the human at risk of developing AD or diagnosed with AD.

Plasma and serum are preferred fluids to be treated with the present method. A preferred fluid is plasma obtained from blood. This plasma is treated with the present method and then optionally combined with previously separated blood cells and returned to the patient. Cerebrospinal fluid may also be treated with the present method.

Apparatus useful in the practice of the present invention are described in the following PCT patent applications which are incorporated herein by reference in their entirety (PCT US/02/19722 (WO 03/000381); PCT US/02/19726 (WO 03/000372) and PCT US/02/19643 (WO 03/000373)). These three applications describe configurations of components in different systems that may be employed to remove lipids and cholesterol from fluids, particularly plasma, and one or more solvents that may be used with these systems.

Accordingly, it is an object of the present invention to provide at least partially delipidated protein and lipoprotein particles that are associated with lipid transport or metabolism.

It is an object of the present invention to provide at least partially delipidated protein and lipoprotein particles that are associated with lipid transport or metabolism comprising one or more of at least partially delipidated HDL, LDL, and VLDL particles.

Another object of the present invention is to provide delipidated particles comprising one or more of delipidated protein and lipoprotein particles that are associated with lipid transport or metabolism, wherein the delipidated particles are effective in preventing, treating and reducing the progression of AD.

It is an object of the present invention to provide a method of preventing, treating and reducing the progression of AD by administering one or more delipidated protein and lipoprotein particles.

Still another object of the present invention is to provide a method of preventing, treating and reducing the progression of AD by decreasing the concentration of lipid and cholesterol within a patient's blood by treating the patient's plasma to decrease the concentration of lipids and cholesterol and form at least partially delipidated protein and lipoprotein particles, and then returning the treated plasma containing at least partially delipidated protein and lipoprotein particles to the vascular system of the patient.

Another object of the present invention is to provide a method of preventing, treating and reducing the progression of AD by decreasing the concentration of lipid and cholesterol within a patient's blood by treating the patient's plasma to decrease the concentration of lipids and cholesterol and form at least partially delipidated protein and lipoprotein particles, optionally combining the treated plasma with the patient's blood cells, and then returning the treated plasma containing at least partially delipidated protein and lipoprotein particles and the patient's blood cells to the vascular system of the patient.

It is an object of the present invention to provide a method of preventing, treating and reducing the progression of AD by decreasing the concentration of lipid and cholesterol within a patient's blood by treating the patient's plasma to decrease the concentration of lipids and cholesterol and form at least partially delipidated protein and lipoprotein particles, and then returning the treated plasma containing at least partially delipidated protein and lipoprotein particles to the vascular system of the patient.

Another object of the present invention to provide a method of preventing, treating and reducing the progression of AD by decreasing the concentration of lipid and cholesterol within a patient's blood by treating the patient's plasma to decrease the concentration of lipids and cholesterol and form at least partially delipidated protein and lipoprotein particles, optionally combining the treated plasma with the patient's blood cells, and then returning the treated plasma containing at least partially delipidated protein and lipoprotein particles and cells to the vascular system of the patient.

It is further an object of the present invention to provide a method of preventing, treating and reducing the progression of AD by that minimizes deleterious effects of solvents on plasma proteins.

It is another object of the present invention to provide a method of preventing or reducing the onset of dementia associated with AD.

Another object of the present invention is to reduce or retard the deposition of amyloid plaque in the brain.

A further object of the invention is to retard the loss of neurons in the brain.

Yet another object of the invention is to affect the proteolytic processing of APP.

Still another object of the invention is to modulate the ratio of Aβ peptides produced in the brain.

Another object of the present invention is to reduce the accumulation and aggregation of Aβ peptides in the brain.

Another object of the invention is to affect the transbilayer distribution of lipid in neurons or glia.

Yet another object of the invention is to affect the distribution of lipid in lipid rafts in neurons or glia.

These and other features and advantages of the present invention will become apparent after review of the following drawings and detailed description of the disclosed embodiments. Various modifications to the stated embodiments will be readily apparent to those of ordinary skill in the art, and the disclosure set forth herein may be applicable to other embodiments and applications without departing from the spirit and scope of the present invention.

### DETAILED DESCRIPTION OF THE INVENTION

Definitions

By the term “fluid” is meant any fluid, including but not limited to, a biological fluid obtained from an organism such as an animal or human. Such biological fluids obtained from an organism include but are not limited to blood, plasma, serum, cerebrospinal fluid, lymphatic fluid, peritoneal fluid, and any other fluid contained within the organism. Blood provides the plasma and serum to be treated with the method of the present invention.

By the terms “first solvent” or “first organic solvent” “or first extraction solvent” are meant a solvent, comprising one or more solvents, used to facilitate extraction of lipid from a fluid. This solvent will enter the fluid and remain in the fluid until being removed. Suitable first extraction solvents include solvents that extract or dissolve lipid, including but not limited to alcohols, hydrocarbons, amines, ethers, and combinations thereof. First extraction solvents may be combinations of alcohols and ethers. First extraction solvents include, but are not limited to n-butanol, di-isopropyl ether (DIPE), diethyl ether, and combinations thereof.

The term “second extraction solvent” is defined as one or more solvents that facilitate the removal of a portion of the first extraction solvent and extracted lipids. Suitable second extraction solvents include any solvent that facilitates removal of the first extraction solvent from the fluid. Second extraction solvents include any solvent that facilitates removal of the first extraction solvent including but not limited to ethers, alcohols, hydrocarbons, amines, and combinations thereof. Second extraction solvents include diethyl ether and di-isopropyl ether, which facilitate the removal of alcohols, such as n-butanol, from the fluid. The term “de-emulsifying agent” is a second extraction solvent that assists in the removal of the first solvent and extracted lipids which may be present in an emulsion in an aqueous layer.

In situations where a second extraction solvent is not required to remove a first solvent, the first solvent may be removed through other means including but not limited to pervaporation or activated charcoal. Pervaporation or activated charcoal may also be employed to remove second extraction solvents.

The term “lipid” is defined as any one or more of a group of fats or fat-like substances occurring in humans or animals. The fats or fat-like substances are characterized by their insolubility in water and solubility in organic solvents. The term “lipid” is known to those of ordinary skill in the art and includes, but is not limited to, complex lipid, simple lipid, triglycerides, fatty acids, glycerophospholipids (phospholipids), true fats such as esters of fatty acids, glycerol, cerebrosides, waxes, and sterols such as cholesterol and ergosterol.

The term “delipidation” refers to the process of removing at least a portion of a total concentration of lipids in a fluid such as plasma and serum. Plasma and serum are used interchangeably herein. The term “delipidation” also refers to removal of lipid from any protein particle or lipoprotein particle capable of binding lipid.

The terms “pharmaceutically acceptable carrier” or “pharmaceutically acceptable vehicle” are used herein to mean any liquid including but not limited to water or saline, a gel, salve, solvent, diluent, fluid ointment base, liposome, micelle, giant micelle, and the like, which is suitable for use in contact with living animal or human tissue without causing adverse physiological responses, and which does not interact with the other components to be administered in a deleterious manner.

The term “patient” refers to animals and humans in this application. Patients are patients at risk of developing, susceptible to, or exhibiting symptoms of AD. Such patients include but are not limited to patients with familial AD and patients with sporadic AD. Patients with trisomy 21 (Down's syndrome) may also be treated with the method of the present invention. All patients with risk factors for developing AD are included within the scope of the present invention. Such patients include but are not limited to those with the following conditions: mutations in the presenilin gene (PS-1), the presenilin gene (PS-2), or the amyloid precursor protein gene (APP) (reviewed in Yankner, 1996); individuals with genes considered risk factors such as the apolipoprotein E (ApoE ε4 variant), α2 macroglobulin, a gene for a component of the αketoglutarate dehydrogenase, the K-variant of butyrylcholinesterase, and several mitochondrial genes; cardiovascular disease; elevated serum cholesterol defined as a blood cholesterol level of greater than 200 mg/dl; individuals with high blood cholesterol levels who have a family history of AD; adults with trisomy 21; individuals with a history of head injury; post menopausal women; individuals over the age of 50 years, particularly over the age of 65 years, and especially over the age of 75 years. Other individuals at risk are described in U.S. Pat. No. 6,472,421 and 6,440,387, and published U.S. Patent Application Pub. No. 2001/0028895. These individuals are all at risk of developing AD. In one embodiment, individuals with these risk factors are treated with the method of the present invention to decrease lipids and cholesterol levels and raise functional levels of HDL prior to developing any mental impairment attributable to AD using accepted neuropsychiatric and diagnostic criteria for probable AD (McKhahn et al. (1984) Neurology 34:939-944). Individuals at risk can be screened using standard blood tests for cholesterol, apoE4, and/or total lipoprotein levels, by performing neuropsychiatric evaluations and by taking a medical and family history using techniques known to one of ordinary skill in the art.

By the term “particle” is meant any particle found in a biological fluid, particularly blood, plasma and serum, that is associated in some way with lipid transport or metabolism. Such particles include protein and lipoprotein particles and are known to one of skill in the art. Such particles include, but are not limited to, HDL, LDL and VLDL. These particles are chemically modified to partially or substantially reduce their lipid content, thereby creating delipidated particles. These delipidated particles are administered to patients at risk of or diagnosed with AD in an amount effective to prevent AD, to delay the progression of AD or to treat AD. These delipidated particles are optionally combined with the patient's blood cells and administered to the patients at risk of or diagnosed with AD.

In another embodiment, these delipidated particles are optionally combined with other therapeutic agents useful in affecting lipid transport and metabolism and/or in preventing AD, slowing progression of AD or treating AD.

The method of the present invention for creating these delipidated particles comprises removing lipid from these particles through a method comprising: obtaining blood containing the lipid, separating the blood cells from the plasma, contacting the plasma with a first organic solvent capable of solubilizing the lipid; and, separating a first phase containing the lipids from a second phase wherein the second phase is substantially free of the lipids. Following preparation of these delipidated particles, they are administered to the patients at risk of developing or diagnosed with AD. Further, these particles are optionally combined with the patient's blood cells before administration to the patient.

Exemplary Solvent Systems for Use in Removal of Lipid From Fluids

The solvent or combinations of solvents to be employed in the process of partially or completely delipidating fluids may be any solvent or combination thereof effective in solubilizing lipids in the fluid. A single solvent system or multiple solvents may be employed. Apparatus useful in the practice of the present invention are described in the following PCT patent applications which are incorporated herein by reference in their entirety (PCT US/02/19722 (WO 03/000381); US/02/19726 (WO 03/000372) and US/02/19643 (WO 03/000373)). These three applications describe configurations of components in different systems that may be employed to remove lipids and cholesterol from fluids, particularly plasma, and one or more solvents that may be used with these systems.

A delipidation process falling within the scope of the present invention uses an optimal combination of energy input and solvent to delipidate fluid. Suitable solvents comprise hydrocarbons, ethers, alcohols, phenols, esters, halohydrocarbons, halocarbons, amines, and mixtures thereof. Aromatic, aliphatic, or alicyclic hydrocarbons may also be used. Other suitable solvents, which may be used with the present invention, include amines and mixtures of amines. One solvent system is DIPE, either concentrated or diluted in water or a buffer such as a physiologically acceptable buffer. One solvent combination comprises alcohols and ethers. Another solvent comprises ether or combinations of ethers, either in the form of symmetrical ethers, asymmetrical ethers or halogenated ethers.

The optimal solvent systems are those that at least partially delipidate the fluid and employ a set of conditions such that there are few or no deleterious effects on the other plasma proteins.

The solvent or combination of solvents preferably have a relatively low boiling point to facilitate removal through a vacuum and possibly heat without destroying other plasma proteins. The solvent or combination of solvents may be employed at a low temperature because heat has deleterious effects on the proteins contained in biological fluids such as plasma. The solvent or combination of solvents are effective to at least partially delipidate the fluid.

Liquid hydrocarbons dissolve compounds of low polarity. Particularly effective are hydrocarbons which are substantially water immiscible. Suitable hydrocarbons include, but are not limited to the following: C₅ to C₂₀ aliphatic hydrocarbons such as petroleum ether, hexane, heptane, octane; haloaliphatic hydrocarbons such as chloroform, 1,1,2-trichloro-1,2,2-trifluoroethane, 1,1,1-trichloroethane, trichloroethylene, tetrachloroethylene, dichloromethane and carbon tetrachloride; thioaliphatic hydrocarbons each of which may be linear, branched or cyclic, saturated or unsaturated; aromatic hydrocarbons such as benzene; alkylarenes such as toluene; haloarenes; haloalkylarenes; and thioarenes. Other suitable solvents may also include saturated or unsaturated heterocyclic compounds such as pyridine and aliphatic, thio- or halo-derivatives thereof.

Suitable esters for use in the present invention include, but are not limited to, ethyl acetate, propylacetate, butylacetate and ethylpropionate. Suitable detergents/surfactants that may be used include but are not limited to the following: sulfates, sulfonates, phosphates (including phospholipids), carboxylates, and sulfosuccinates. Some anionic amphiphilic materials useful with the present invention include but are not limited to the following: sodium dodecyl sulfate (SDS), sodium decyl sulfate, bis-(2-ethylhexyl) sodium sulfosuccinate (AOT), cholesterol sulfate and sodium laurate.

Solvents may be removed from delipidated fluids through the use of additional solvents. For example, demulsifying agents such as ethers may be used to remove a first solvent such as an alcohol from an emulsion. Removal of solvents may also be accomplished through other methods, which do not employ additional solvents, including but not limited to the use of activated charcoal. Activated charcoal may be used in a slurry or alternatively, in a column to which a mixture is applied. Pervaporation may also be employed to remove one or more solvents from delipidated fluids.

Examples of suitable amines for use in removal of lipid from lipid-containing fluids, such as plasma, in the present invention are those which are substantially immiscible in water. Typical amines are aliphatic amines—those having a carbon chain of at least 6 carbon atoms. A non-limiting example of such an amine is C₆H₁₃NH₂.

Ether is a solvent useful in the method of the present invention, especially the C₄-C₈ containing-ethers, including but not limited to ethyl ether, diethyl ether, and propyl ethers (including but not limited to di-isopropyl ether). Asymmetrical ethers may also be employed. Halogenated symmetrical and asymmetrical ethers may also be employed.

Low concentrations of ethers may be employed to remove lipids when used alone and not in combination with other solvents. For example, a low concentration range of ethers include 0.5% to 30%. Such concentrations of ethers that may be employed include, but are not limited to the following: 0.5%, 0.625%, 1.0% 1.25%, 2.5%, 5.0% and 10% or higher. It has been observed that dilute solutions of ethers are effective. Such solutions may be aqueous solutions or solutions in aqueous buffers, such as phosphate buffered saline (PBS). Other physiological buffers may be used, including but not limited to bicarbonate, citrate, Tris, Tris/EDTA, and Trizma. In one embodiment, the ethers are di-isopropyl ether (DIPE) and diethyl ether (DEE).

High concentrations of ethers may also be employed to remove lipids from fluids such as plasma. In some cases, 100% DIPE has effectively removed lipids from plasma without adverse effects on proteins.

When alcohols are used alone, appropriate alcohols are those which are not appreciably miscible with plasma or other biological fluids. Alcohols which may be used include, but are not limited to, straight chain and branched chain alcohols, including butanols, pentanols, hexanols, heptanols, octanols and those alcohols containing higher numbers of carbons. In some cases, 100% n-butanol has effectively removed lipids from plasma without adverse effects on proteins.

When alcohols are used in combination with another solvent, for example, an ether, a hydrocarbon, an amine, or a combination thereof, C₁-C₈ containing alcohols may be used. Alcohols for use in combination with another solvent include C₄-C₈ containing alcohols. Accordingly, alcohols that fall within the scope of the present invention are butanols, pentanols, hexanols, heptanols and octanols, and iso forms thereof, in particular, C₄ alcohols or butanols (1-butanol and 2-butanol). The specific alcohol choice is dependent on the second solvent employed.

Ethers and alcohols can be used in combination as a first solvent for treating the fluid containing lipid. Any combination of alcohol and ether may be used provided the combination is effective to at least partially remove lipid from the fluid, without having deleterious effects on the plasma proteins. In one embodiment, lipid is partially or substantially removed from the plasma. When alcohols and ether are combined as a first solvent for removing lipid contained in a fluid, ratios of alcohol to ether in this solvent are about 0.01%-60% alcohol to about 40%-99.99% of ether, with a specific ratio of about 10%-50% of alcohol with about 50%-90% of ether, with a more specific ratio of about 20%-45% alcohol and about 55%-80% ether.

One combination of alcohol and ether is the combination of butanol and di-isopropyl ether (DIPE). When butanol and DIPE are combined as a first solvent for treating the fluid, ratios of butanol to DIPE in this solvent are about 0.01%-60% butanol to about 40%-99.99% of DIPE, with a specific ratio of about 10%-50% of butanol with about 50%-90% of DIPE, with a more specific ratio of about 20%-45% butanol and about 55%-80% DIPE.

Another combination of alcohol and ether is the combination of butanol with diethyl ether (DEE). When butanol is used in combination with DEE as a first solvent, ratios of butanol to DEE are about 0.01%-60% butanol to about 40%-99.99% of DEE, with a more specific ratio of about 10%-50% of butanol with about 50%-90% of DEE, with a most specific ratio of about 20%-45% butanol and about 55%-80% DEE. One specific ratio of butanol and DEE in a first solvent is about 40% butanol and about 60% DEE. This combination of about 40% butanol and about 60% DEE (vol:vol) has been shown to have no significant effect on a variety of biochemical and hematological blood parameters, as shown for example in U.S. Pat. No. 4,895,558.

Isoflurane, sevoflurane or DIPE have been combined individually with n-butanol. These combinations enhanced the solubility of n-butanol in plasma. These combinations delipidated plasma as well as plasma proteins and plasma lipoproteins.

Biological Fluids and Treatment Thereof for Reducing Lipid Levels and Forming Delipidated Particles

As stated above, various biological fluids may be treated with the method of the present invention in order to reduce the levels of lipid in the biological fluid and create delipidated particles useful to prevent, treat or generally retard the progression of AD. In one embodiment, plasma obtained from blood of an animal or human is treated with the method of the present invention in order to reduce the concentration of lipid within the plasma and to produce delipidated protein and lipoprotein particles, such as delipidated HDL, LDL and VLDL particles. In this embodiment, plasma may be obtained from an animal or human patient by withdrawing blood from the patient using well-known methods and treating the blood in order to separate the cellular components of the blood (red cells, white cells and platelets) from the plasma. Such methods for treating the blood are known to one of ordinary skill in the art and include but are not limited to centrifugation and filtration. One of ordinary skill in the art understands the proper centrifugation conditions for separating red cells, white cells and platelets from the lipid-containing plasma. Filtration may include diafiltration or filtration through membranes with pore sizes that separate the lipid-containing plasma, from the red and white cells and platelets. Use of the present invention permits treatment of lipid-containing plasma, without having deleterious effects on other plasma proteins.

Treatment of lipid-containing biological fluids other than blood does not generally involve separation of the cells from the fluid prior to initiation of the delipidation procedure.

Once a biological fluid, such as plasma, is obtained either in this manner, or for example, from a storage facility housing bags of plasma, the plasma is contacted with a first organic solvent, as described above, capable of solubilizing lipid in the lipid-containing biological fluid. The first organic solvent is combined with the plasma in a ratio wherein the first solvent is present in an amount effective to substantially solubilize the lipid in the fluid. Exemplary ratios of first solvent to plasma (expressed as a ratio of first organic solvent to plasma) are described in the following ranges: 0.5-4.0:0.5-4.0; 0.8-3.0:0.8-3.0; and 1-2:0.8-1.5. Various other ratios may be applied, depending on the nature of the biological fluid. For example, in the case of cell culture fluid, the following ranges may be employed of first organic solvent to cell culture fluid: 0.5-4.0:0.5-4.0; 0.8-3.0:0.8-3.0; and 1-2:0.8-1.5.

After contacting the fluid containing the lipid with the first solvent as described above, the first solvent and fluid are mixed, using methods including but not limited to one of the following suitable mixing methods: gentle stirring; vigorous stirring; vortexing; swirling; homogenization; and, end-over-end rotation.

The amount of time required for adequate mixing of the first solvent with the fluid is related to the mixing method employed. Fluids are mixed for a period of time sufficient to permit intimate contact between the organic and aqueous phases, and for the first solvent to at least partially or completely solubilize the lipid contained in the fluid. Typically, mixing will occur for a period of about 10 seconds to about 24 hours, possibly about 10 seconds to about 2 hours, possibly approximately 10 seconds to approximately 10 minutes, or possibly about 30 seconds to about 1 hour, depending on the mixing method employed. Non-limiting examples of mixing durations associated with different methods include 1) gentle stirring and end-over-end rotation for a period of about 10 seconds to about 24 hours, 2) vigorous stirring and vortexing for a period of about 10 seconds to about 30 minutes, 3) swirling for a period of about 10 seconds to about 2 hours, or 4) homogenization for a period of about 10 seconds to about 10 minutes. Static mixing methods in static mixing tubes may also be employed.

Separation of Solvents

After mixing of the first solvent with the fluid, the solvent is separated from the fluid being treated. The organic and aqueous phases may be separated by any suitable manner known to one of ordinary skill in the art. Since the first solvent is typically immiscible in the aqueous fluid, the two layers are permitted to separate and the undesired layer is removed. The undesired layer is the solvent layer containing dissolved lipids and its identification, as known to one of ordinary skill in the art, depends on whether the solvent is more or less dense than the aqueous phase. An advantage of separation in this manner is that dissolved lipids in the solvent layer may be removed.

In addition, separation may be achieved through means, including but not limited to the following: removing the undesired layer via pipetting; centrifugation followed by removal of the layer to be separated; creating a path or hole in the bottom of the tube containing the layers and permitting the lower layer to pass through; utilization of a container with valves or ports located at specific lengths along the long axis of the container to facilitate access to and removal of specific layers; by permitting the layers to settle over time, and any other means known to one of ordinary skill in the art. Another method of separating the layers, especially when the solvent layer is volatile, is through distillation under reduced pressure or evaporation at room temperature, optionally combined with mild heating. In one embodiment employing centrifugation, relatively low g forces are employed, such as 900×g for about 5 to 15 minutes to separate the phases. Centrifugation and gravity may be used to achieve bulk separation of the layers.

Another method of removing solvent is through the use of activated charcoal. This activated charcoal is optionally contained in a column. Alternatively the activated charcoal may be used in slurry form. Various biocompatible forms of activated charcoal may be used in these columns. Pervaporation methods and use of activated charcoal to remove solvents are methods useful for removing solvent in the practice of the present invention. Solvent may also be removed by passing nitrogen or a gas stream, such as an air gas stream, over the surface of the solvent.

Following separation of the first solvent from the treated fluid, some of the first solvent may remain entrapped in the aqueous layer as an emulsion. Optionally, a de-emulsifying agent is employed to facilitate removal of the trapped first solvent. The de-emulsifying agent may be any agent effective to facilitate removal of the first solvent. A useful de-emulsifying agent is ether. A useful de-emulsifying ether is diethyl ether. The de-emulsifying agent may be added to the fluid or in the alternative the fluid may be dispersed in the de-emulsifying agent. Alkanes in a ratio of about 0.5 to 4.0 to about 1 part of emulsion (vol:vol) may be employed as a de-emulsifying agent, followed by washing to remove the residual alkane from the remaining delipidated fluid. Alkanes include, but are not limited to, pentane, hexane and higher order straight and branched chain alkanes.

The de-emulsifying agent, such as ether, may be removed through means known to one of skill in the art, including such means as described in the previous paragraph. One convenient method to remove the de-emulsifying agent, such as ether, from the system, is to permit the ether to evaporate from the system in a running fume hood or other suitable device for collecting and removing the de-emulsifying agent from the environment. In addition, de-emulsifying agents may be removed through application of higher temperatures, for example from about 24 to 37° C. with or without pressures of about 10 to 20 mbar. Another method to remove the de-emulsifying agent involves separation by centrifugation, followed by removal of organic solvent through aspiration, further followed by evaporation under reduced pressure (for example 50 mbar) or further supply of an inert gas, such as nitrogen, over the meniscus to aid in evaporation. Yet another method of removing a first solvent or a demulsifying agent is through the use of adsorbants, such as activated charcoal. This activated charcoal is optionally contained in a column, as described above. Still another method of removing solvent is the use of hollow fiber contactors. Pervaporation methods and activated charcoal adsorbant methods of removing solvents are useful methods.

Methods of Treating Biological Fluids (Delipidation)

It is to be understood that the method of the present invention may be employed in either a continuous or discontinuous manner. That is, in a continuous manner, a fluid may be fed to a system employing a first solvent which is then mixed with the fluid, separated, and optionally further removed through application of a de-emulsifying agent. The continuous method also facilitates subsequent return of the delipidated fluid to a desired location. Such locations may be containers for receipt and/or storage of such treated fluid, and may also include the vascular system of a human or animal or some other body compartment of a human or animal, such as the pleural, pericardial, peritoneal, and abdominopelvic spaces.

In one embodiment of the continuous method of the present invention, a biological fluid, for example, blood, is removed from an animal or a human through means known to one of ordinary skill in the art, such as a catheter. Appropriate anti-clotting factors as known to one of ordinary skill in the art are employed, such as heparin, anticoagulant citrate dextrose solution, formula A (ACDA), ethylenediaminetetraacetic acid (EDTA) or citrate. This blood is then separated into its cellular and plasma components through appropriate means such as a filter, a spinning membrane or a centrifuge. The plasma is then contacted with the first solvent and mixed with the first solvent to effectuate lipid removal from the plasma. Following separation of the first solvent from the treated plasma, a de-emulsifying agent is optionally employed to remove entrapped first solvent. After ensuring that acceptable levels (non-toxic) of first solvent or de-emulsifying agent, if employed, are found within the plasma, the plasma is then optionally combined with the cells previously separated from the blood to form a new blood sample containing at least partially delipidated plasma.

Through the practice of this method, the lipid content of the fluid, especially plasma is reduced, and delipidated protein and lipoprotein particles are formed. Following recombination with the cells originally separated from the blood, this sample may be reintroduced into either the vascular system or some other system of the human or animal. The effect of such treatment of plasma removed from the human or animal and return of the sample containing the partially or completely delipidated plasma, to the human or animal causes a net decrease in the concentration of lipid within the vascular system of the human or animal. In this mode of operation, the method of the present invention is employed to treat body fluids in a continuous manner—while the human or animal is connected to an extracorporeal device for such treatment.

In yet another embodiment, the discontinuous or batch mode, the human or animal is not connected to an extracorporeal device for processing bodily fluids with the method of the present invention. In a discontinuous mode of operation, the present invention employs a fluid previously obtained from a human or animal, which may include, but is not limited to, blood, plasma or serum. If the sample is blood, the blood cells are separated before the delipidation procedure is performed. The sample may be contained within a blood bank or in the alternative, drawn from a human or animal prior to application of the method. In this mode of operation, this sample is treated with the method of the present invention to produce a new sample which contains reduced levels of lipid and partially delipidated protein and lipoprotein particles. One embodiment of this mode of the present invention is to treat plasma samples previously obtained from animals or humans and stored in a blood bank for subsequent transfusion. These samples may be treated with the method of the present invention to minimize or eliminate transfusion of a plasma containing high lipid levels.

Frequency of Plasma Delipidation Treatment

Administration of the delipidated particles of the present invention is performed in an amount and with a frequency that is effective to treat, prevent, or delay the progression of AD.

Delipidation and Administration of Delipidated Particles in Combination with Other Therapies for Modulating Lipid Metabolism and Treating or Preventing AD

The present invention provides novel particles comprising at least partially delipidated protein and lipoprotein particles useful for preventing, delaying the progression of and treating AD. The present invention also provides methods for making and using these particles and for administration of these particles together with delipidated plasma for preventing, delaying the progression of and treating AD. The present methods may be used in conjunction with other treatments known to one of skill in the art for reducing cholesterol and LDL, and for increasing HDL. The present invention may also be used in conjunction with other therapeutic treatments for AD known to one of ordinary skill in the art. These combination therapies may be administered in accordance with standard regimens known to one of ordinary skill in the art and may occur before, during or after the delipidation procedure and administration of the delipidated particles. The following paragraphs describe some of these treatments.

Administration of Compounds Which function as HDL

Compounds which function as HDL include synthetic HDL which contains lipid such as phosphatidyl choline, phosphatidyl serine, phosphatidyl ethanolamine, and other phospholipids and may be used in combination therapy with the present invention. Compounds which enhance HDL function include HDL associated proteins such as apo A1 or variants thereof including apo AI-Milano and biologically active peptides derived therefrom, reverse lipid transport (RLT) peptides, apoe, enzymes associated with HDL such as paraoxonase, and LCAT, alone or, more preferably, formulated in combination with liposomes or emulsions. These compositions can also be administered with compounds that increase HDL levels specifically, and thereby improve the HDL cholesterol to total cholesterol ratio or the apoA-I to total cholesterol ratio, and/or with compositions which are effective to improve the HDL or apoA-I to total blood cholesterol levels. Alternatively, or in addition, cholesteryl ester transfer protein inhibitors (CETP inhibitors) can be administered to the patients to treat or prevent AD.

Therapy Involving Both Decrease in Plasma Lipids and Cholesterol Using the Present Invention in Conjunction with Statins with or Without Anti-Inflammatories

It is to be understood that the lipid reduction procedures of the present invention may be combined with other therapies for prevention, amelioration or treatment of AD. Such therapies include but are not limited to the following: estrogen replacement therapy; acetylcholinesterase inhibitors (tacrine, donepezil and rivastigmine); monoamine oxidase inhibitors (e.g., selegiline); antioxidants (e.g., vitamin E, vitamin C); anti-inflammatory drugs (such as non-steroidal anti-inflammatory drugs) and are commonly known to neurologists skilled in the art. Additional therapies that may be combined with the method of the present invention include the use of lipid lowering agents called statins. Some additional therapies are found in U.S. non-provisional patent application publication number 2001/0028895, incorporated herein in its entirety.

Compositions to Decrease Production of Aβ

Administration of synthetic HDL or compounds that enhance HDL can be used in conjunction with the compositions and methods of the present invention to decrease production of Aβ, thereby decreasing the risk of developing AD. The same methods can also be used to treat patients who have already been diagnosed with AD. The synthetic HDL or compounds which enhance HDL function can also be administered with compounds which increase HDL cholesterol or apoA-I levels, such as CETP inhibitors. These can also be administered in combination with agents which lower LDL levels, for example, HMG CoA reductase inhibitors or compounds, such as intestinal cholesterol absorption inhibitors (e.g. beta-sitosterol, acylCoA:cholesterol acyltransferase (ACAT) inhibitors, saponins), bile acid sequestrants, fibrates, or niacin (nicotinic acid).

Synthetic HDL

Compositions which function as HDL, thereby effectively increasing HDL blood levels, include liposomal formulations as described in WO 95/23592 by the University of British Columbia and can be used in conjunction with the compositions and methods of the present invention. Some of these are formed of phospholipids, such as sphingomyelin, phosphatidyl choline, phosphatidyl serine, and phosphatidyl ethanolamine, alone or in combination. Liposomes of about 125 nm.+/−0.50 nm (i.e., large unilamellar liposomes) are useful, although larger and smaller liposomes may also be useful.

Compositions Which Increase HDL Function.

Compositions which enhance HDL function include apo AI or variants thereof including Apo AI-Milano and biologically active amphipathic peptides derived therefrom, alone or in combination with liposomes or emulsions, for examples, as described in U.S. Pat. No. 5,876,968, and references cited therein, and can be used in conjunction with the compositions and methods of the present invention.

Suitable apo A and apo A variant compositions are described in EP 0469017 by Pharmacia Upjohn, EP 067703 by Farmatolia, and U.S. Pat. No. 5,834,596 to Ageland, et al. Proapolipoprotein AI is described in U.S. Pat. No. 5,059,528 to Bollen, et al. Synthetic amphipathic peptides are described in PCT/US00/8788 by Dasseaux, et al. Peptide/lipid complexes are described in PCT/US98/20330 by Dasseaux. Either compounds are described in PCT/US00/8799 by Esperion Therapeutics.

Human apolipoprotein A-I (apo A-I) possesses multiple tandem repeating 22-mer amphipathic alpha-helixes. Computer analysis and studies of model synthetic peptides and recombinant protein-lipid complexes of phospholipids have suggested that apo A-I interacts with HDL surface lipids through cooperation among its individual amphipathic helical domains. Lipid-associating properties of apo A-I are localized to the N- and C-terminal amphipathic domains and may be used in the present invention. N- and C-terminal peptides (44-65 and 220-241) of apo A-I may be used.

Plasma Cholesterol Level Lowering Agents and Plasma Triglyceride Level Lowering Agents

These delipidation methods of the present invention may be used in combination with plasma cholesterol level lowering agents and plasma triglyceride level lowering agents to prevent, retard the progression of or treat AD. These agents include HMG CoA reductase inhibitors, bile acid sequestrants, agents that block intestinal cholesterol absorption, saponins, neomycin, and acyl CoA:cholesterol acyl transferase inhibitors.

Representative HMG CoA reductase inhibitors include the statins, including lovastatin, simvastatin, compactin, fluvastatin, atorvastatin, cerivastatin, and pravastin. Representative fibrates include clofibrate, fenofibrate, gemfibrozil, or bezafibrate. Compounds which inhibit cholesterol biosynthetic enzymes, including 2,3-oxidosqualene cyclase, squalene synthase, and 7-dehydrocholesterol reductase, can also be used. Representative compositions which decrease uptake of dietary cholesterol include the bile acid binding resins (cholestryramine and colestipol). Probucol, nicotinic acid, garlic and garlic derivatives, and psyllium are also used to lower blood cholesterol levels. Probucol and the fibrates increase the metabolism of cholesterol containing lipoproteins. Plasma triglyceride lowering agents also include niacin, carboyxalkylethers, thiazolidinediones, eicosapentanoic acid, EPA, and acylCoA:cholesteryl acyltransferase (ACAT).

Cholesteryl Ester Transfer Protein (CETP) Inhibitors

Patients receiving the delipidation therapy of the present invention can also be treated with CETP inhibitors, alone or in combination with the compositions which act as HDL or act to enhance HDL function. Representative compounds include PD 140195 as described by Bisgaier, et al., LIPIDS 29(12), 811-818 (1994); tetrahydroquinoline derivatives described in EPA 987251 by Pfizer, pyridine derivatives described in DE 19731609-C3 by Searle & Co.; triazole derivates described in WO 99/14204 by Searle & Co; substituted tetrahydro-napthalene derivates described in DE 741050 by Bayer A G; benzyl-biphenyl derivatives described in DE 741400 by Bayer A G; tetrahydro-quinoline derivatives described by Bayer A G phenylamine derivatives described by JP 11049743 by Japan Tobacco Inc.; erabulenols described by Tomoda, et al., J. Antibiotics 51(7), 618-623 (1998); BM99-1 and BM99-2 described by JP09059155 by Kaken Pharm Co Ltd.; tetracyclic catechols as described by Xia, et al., 212.sup.th Amer. Chem. Soc. Nat. Meeting, Orlando, Fla. Aug. 25-29, 1996; and vaccines, described in WO 99/20302 by Rittershaus; Rittershaus, et al., Arterioscler. Thromb. Vasc. Biol. 20:2106-2112 (2000); WO 99/15655 by Monsanto; and WO 9741227 by T Cell Science. Antisense is described in DE 19731609 by Boehringer Ingelheim Pharm KG.

Methods of Treatment

These compositions described above, used in conjunction with the methods and compositions of the present invention are typically administered orally, in tablet form, once daily, using the same or lower dosages as are currently used to treat atherosclerosis. Lower dosages would more typically be used when the treatment is prophylactic. As noted above, some compositions, such as the liposomes, and emulsions of compounds enhancing HDL function, will more typically be administered by means of injection. Several administration schedules are provided in U.S. non-provisional patent application publication number 2001/0028895.

Compositions are administered in an amount and for a length of time effective to increase relative HDL to total cholesterol levels sufficient to decrease deposition of plaque in the brains of patients at risk of developing AD. The increase can be due to the administration of the “synthetic” HDL or to enhancement of function of the endogenous HDL.

The compositions can be administered in a single or multiple dosages. For multiple administration, the compositions for IV infusion are given usually once a week, however they may be given every two to four days up to once every year. An effective dose and treatment regimen is given to block the onset of AD or to treat AD and can be assessed by periodic evaluations of the patient. Clinical diagnosis can be performed by interview with the subject and relatives with questionnaire techniques familiar to those skilled in the evaluation of conditions of dementia.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### EXAMPLE 1

Reduction in Cholesterol Associated with HDL and LDL Following the Delipidation Procedure in Pig Plasma and Reduction in Circulating Cholesterol Associated with HDL and LDL in Pigs Following Infusion of the Delipidated HDL and LDL Particles

Blood was obtained from a pig, plasma separated from blood cells, and both the blood cells and numerous biochemical parameters of the plasma were characterized using standard blood chemistry techniques and assays known to one of ordinary skill in the art. These parameters included but were not limited to total cholesterol (TC), triglycerides (TG), cholesterol associated with high density lipoprotein (HDL), and cholesterol associated with low density lipoprotein (LDL).

A minimum of 120 mL of pig plasma was placed in a Schott bottle, solvent was added and mixed with the plasma through end over end rotation at 30 rpm for 15 minutes. Several 120 ml aliquots of pig plasma were processed in this manner. The solvent employed was a mixture of n-butanol to DIPE at a ratio of 25 n-butanol:75 DIPE. The ratio of solvent to plasma was 0.5 parts solvent to 1 part plasma. The bottles were centrifuged at 1000×g for 10 minutes. The bottom layer (plasma layer) was removed through vacuum aspiration using a pump and applied to an activated charcoal column (Asahi Hemosorba CH350 column). This column was previously primed with dextrose followed by saline. The plasma was then pumped through the charcoal column at 50 ml/min. This sample was infused into the pig that produced the original plasma sample.

The data from two pigs is shown in Table 1. The data indicate that the delipidation procedure dramatically reduced TC, TG, cholesterol associated with HDL, and cholesterol associated with LDL in the plasma samples (referred to as post delipidation/post charcoal plasma). Following infusion into individual pigs (indicated as # 1 or #2) of this delipidated sample containing the HDL and LDL particles with greatly reduced cholesterol, the levels of TC, TG, cholesterol associated with HDL, and cholesterol associated with LDL in the plasma were reduced (compare pig #1 pre- vs post-infusion and pig #2 pre- vs post-infusion). Taken together, these data indicate that the delipidation procedure and administration of the delipidated sample containing delipidated HDL and LDL particles is effective in reducing the levels of cholesterol, lipids and lipoproteins involved in lipid transport and metabolism.

TABLE 1

| | | TC | TG | HDL | LDL |
|---|---|---|---|---|---|
| | | mg/dL | mg/dL | mg/dL | mg/dL |
| Pre-delipidation plasma | #1 | 73 | 32 | 40 | 27 |
| | #2 | 87 | 26 | 37 | 45 |
| Post delipidation/ | #1 | 2 | 4 | 0 | 1 |
| post charcoal plasma | #2 | 1 | 3 | 0 | 0 |
| Pig pre-reinfusion | #1 | 92 | 36 | 39 | 46 |
| | #2 | 108 | 28 | 37 | 65 |
| Pig post reinfusion | #1 | 80 | 21 | 35 | 41 |
| | #2 | 93 | 24 | 31 | 57 |

TC is total cholesterol, TG is triglycerides, HDL is cholesterol associated with high density lipoprotein, LDL is cholesterol associated with low density lipoprotein

### EXAMPLE 2

Effects of Plasma Delipidation on Atherosclerosis in ApoE−/− Mice

After one week of quarantine, thirty apoE−/− male mice, weighing approximately 20-25 grams and 7-8 weeks of age, are divided into a treatment group and a control group and fed high cholesterol food throughout the experimental period. ApoE−/−mice have been show to develop spontaneous atherosclerotic lesions that are similar to lesions in humans and high cholesterol food accelerates the formation of these plaques in apoE−/− mice. Twenty-five percent (25%) of the total blood volume of each mouse in the treatment group is collected and delipidated one time per week for six weeks. The mice in the treatment group (Group 2) receive a total of six treatments over a six week time period. As demonstrated in Table 2, the control group (Group 1) is subjected to collection of blood and return into each mouse.

TABLE 2

| Group | Animal | Number | Bleeding | Delipidation | Duration |
|---|---|---|---|---|---|
| 1 | 1-15 | 15 | Yes | No | 6 weeks |
| 2 | 16-30 | 15 | Yes | Yes | 6 weeks |

For each collection, the mice are anesthetized by inhalation of isoflurane. The blood is collected via orbital sinus with a micro blood collecting tube or through a jugular vein cannula. The average volume of blood collected is about 300-400 μL and citrate is used as an anti-coagulant at a ratio of 1:10.

Plasma and blood cells are isolated from blood at room temperature. Plasma is pooled and subjected to delipidation. Blood cells are stored at room temperature. Plasma is administered via tail vein injection.

Plasma and blood cells are isolated, and plasma is pooled in a glass tube. Solvents (60%:40% v/v diisopropyl ether:n-butanol) saturated with sterile 0.9% sodium chloride solution are added to the plasma in a 2:1 ratio. The solvent:plasma mixture is rotated end-over-end for 20 minutes at 30 rpm and then centrifuged at 1000 g for 2 minutes. The top phase or solvent layer is then removed. Residual butanol is removed by washing with diethyl ether. The residual diethyl ether is removed from the delipidated plasma by blowing nitrogen on the meniscus at room temperature. Pooled delipidated plasma is mixed with pooled blood cells from which the plasma was obtained, the mixture was divided and infused into the mice by tail vein injection or orbital sinus injection. The steps are completed at room temperature.

Tissue Preparation

Animals are harvested after six treatments over six weeks. Mice are anesthetized by pentobarbital (50 mg/kg IP). The inferior vena cava is exposed, and a blood sample is collected into a 1 ml syringe containing citrate (1:10 used as an anti-coagulant) through a 25 guage needle. Plasma is isolated by centrifugation at 2000 rpm for 15 min at 4° C. Plasma is finally transferred into an Eppendorf tube marked with animal # and date and stored at −80° C. The heart is exposed and perfused with PBS to clear the blood, and perfusion-fixed with 4% paraformaldehyde in PBS (pH 7.4) for 5 min. The aorta and both carotid arteries are removed and placed in 4% paraformaldehyde in PBS for 4-16 hours. The samples are finally stored in 70% ethanol.

The artery is carefully removed from adjacent tissues (e.g. the adipose tissue) to avoid non-specific staining with oil red O. The artery is processed for oil red-O staining and analyzed by computer-assisted planimetry to measure the surface areas of atherosclerotic lesions. The measurements are performed in a blinded fashion to exclude bias.

Brain tissue is also harvested and prepared for staining of amyloid plaque, neurofibrillary tangles, phosphorylated tau protein and various forms of Aβ.

The stain solution is prepared with 0.2% Oil Red-O (Sigma) in methanol and 1 M NaOH. The adipose tissue is carefully removed from the artery and the artery is cut longitudinally. The tissue is stained with the solution in 1.5-2 ml centrifuge tube for 50 minutes at room temperature by gently mixing. The tissue is washed with 70% ethanol for 30 minutes. The tissue is transferred to distilled water.

The tissue is processed for histological analysis after oil red-O staining is complete. Four evenly-spaced cross sections are prepared and subjected to hematoxylin-eosin stain. All cross sections are 5 μm thick. Intima and media areas of each cross section are determined by computer-assisted planimetry, and the mean intima and media cross-sectional area is calculated for each artery. Measurements are performed in a blinded fashion to exclude bias. The percentage of lesions is calculated according to the following formula: lesion percentage=lesion areas/whole areas of artery.

Anti-BrdU staining is performed with a BrdU staining kit (Zymed Laboratories) to identify proliferating cells. A rat monoclonal antibody to Mac-3 (Pharmingen) is used to identify macrophages. Smooth muscle α-actin staining is performed with anti-human smooth muscle α-actin monoclonal antibody (clone 1A4, Dako). von Willebrand factor (vWF) is detected with rabbit anti-human vWF antibody. Tissue factor expression is detected by digoxigenin-labelled human factor VIIa staining. The chromogen for tissue factor staining is nitro blue tetrazolium chloride/X-phosphate (Digoxigenin Detection Kit, Boehringer Mannheim), and counterstaining is performed with nuclear fast red solution (Poly Scientific R&D Corp).

In the plasma and arterial tissue, total cholesterol, free cholesterol, triglyceride, apoAI, apoB, and apoE are measured with WAKO kits supplied by Wako Diagnostics. In the brain tissue, Aβ, senile plaques, and intracellular neurofibrillary tangles are examined.

Arterial tissue harvested from the treatment group shows fewer lesions and has a lower lesion percentage than tissue harvested from the control group. Similarly, brain tissue harvested from the treatment group shows fewer senile plaques, less neurofibrillary tangles, lower levels of phosphorylated tau, and Aβ than brain tissue harvested from the control group.

### EXAMPLE 3

Fifty (50) patients diagnosed with AD undergo plasma delipidation two times per week for three weeks and administration of delipidated plasma and protein and lipoprotein particles. Fifty patients with AD do not undergo plasma delipidation. The patients in the treatment group exhibit improvement and reduction of symptoms of dementia compared to patients in the control group.

### EXAMPLE 4

Fifty (50) patients ranging in age from 50-60 and having high cholesterol levels (more than 350 mg/dl) undergo plasma delipidation two times per week for six months and administration of delipidated plasma and protein and lipoprotein particles. Fifty patients ranging in age from 50-60 and having high cholesterol levels (more than 350 mg/dl) do not undergo plasma delipidation. The patients are followed for ten years. Surviving patients in the treatment group exhibit a delayed onset of symptoms of AD compared to patients in the control group.

### EXAMPLE 5

Forty (40) patients with a familial history of AD, and possessing the apolipoprotein E (ApoE epsilon 4) gene variant undergo plasma delipidation once per week for twelve months and administration of delipidated plasma and protein and lipoprotein particles. Forty patients with a familial history of AD, and possessing the apolipoprotein E (ApoE epsilon 4) gene variant, do not undergo plasma delipidation. The patients are followed for ten years. Patients in the treatment group exhibit a delayed onset of symptoms of AD compared to patients in the control group.

### EXAMPLE 6

Sixty (60) patients having high cholesterol levels (more than 350 mg/dl) undergo plasma delipidation two times per week for three months and administration of delipidated plasma and protein and lipoprotein particles. These patients are divided into four groups of 15 per group and also receive either 10 mg, 20 mg, 40 mg or 80 mg of a statin drug once per day in the evening. Sixty patients having high cholesterol levels (more than 350 mg/dl) do not undergo plasma delipidation but are divided into four groups of 15 and also receive either 10 mg, 20 mg, 40 mg or 80 mg of a statin drug once per day in the evening (control groups). The patients are followed for ten years. Patients in the treatment groups exhibit a delayed onset of symptoms of AD compared to patients in the control groups.

### EXAMPLE 7

Measurement of ApoE, Aβ and Lipid in Plasma and CSF of Rhesus Monkeys Before and After Decreasing Lipid Levels in Plasma

Rhesus monkeys are anesthetized. Blood samples are removed through venipuncture and CSF samples are obtained from a lumbar tap in accordance with proper and accepted techniques known to one of ordinary skill in the art. The blood samples are processed to measure the levels of the following parameters in the plasma: LDL, VLDL, HDL, lipids, cholesterol, 24S-cholesterol, triglycerides. The CSF samples are processed to measure the levels of the following parameters: lipid, 24S-cholesterol, ApoE, Aβ and its sub forms including Aβ40, Aβ42 and Aβ43. These molecules are measured using techniques known to one of ordinary skill in the art.

These plasma and CSF parameters establish a baseline for comparison to measurement of these parameters after the monkeys are subjected to the delipidation procedure. After a suitable period of recovery from the plasma and CSF withdrawal, rhesus monkeys are anesthetized. Venous blood is withdrawn via syringe and treated with the delipidation procedure. Following delipidation of the plasma, the delipidated plasma including the at least partially delipidated protein and lipoprotein particles, is combined with the red cells, white cells and platelets and returned to the animal through a vascular catheter. The procedure is performed a minimum of four times.

Blood samples and CSF samples are withdrawn from these rhesus monkeys at various intervals after the delipidation procedures are complete. Analysis of the plasma and CSF parameters listed above shows that over time, plasma levels of lipid, total cholesterol, of ApoE, and Aβ, and CSF levels of Aβ and ApoE decline in a manner consistent with a reduction in AD pathology.

### EXAMPLE 8

Measurement of ApoE and Aβ in Cerebral Cortex and Hippocampal Cortex of Rhesus Monkeys Following Reduction in Plasma Lipid Levels

Blood and CSF samples are obtained to measure the baseline parameters described in Example 7 above. Rhesus monkeys are subjected to the plasma delipidation procedure for a number of sessions that is effective to affect cerebral cortical and hippocampal cortical levels of Aβ and its subforms Aβ40 and Aβ42, and also levels of ApoE.

Other monkeys receive a control procedure in which blood is removed, cells are separated from plasma but the plasma is not treated with solvents to remove lipids. The treated or untreated plasma samples are combined with the blood cells that are separated and then returned to the same monkey from which they are obtained.

Following several delipidation or control treatments, the control and experimental monkeys in each group are anesthetized, blood and CSF samples are withdrawn for analysis of relevant parameters, and the animals are sacrificed through drug overdose with the appropriate agents as known to one of ordinary skill in the art. The calvarium is rapidly removed from each animal and the brain is dissected free of the dura and cranial nerves. The brain is blocked in to several coronal slices, frozen and later sectioned coronally in a cryostat. Selected regions of the cerebral cortex (frontal, temporal, parietal and occipital) and hippocampal cortex are isolated and tissue samples are obtained. The samples are processed for measurement of ApoE, Aβ and its subforms Aβ40 and Aβ42 using assays routinely available to one of ordinary skill in the measurement of this apolipoprotein and these peptides. Protein levels in these samples are measured using standard assays such as the Bradford assay. Levels of ApoE, Aβ and its subforms Aβ40 and Aβ42, are expressed in terms of protein levels in the samples.

Results demonstrate that cerebral cortical and hippocampal cortical levels of Aβ and its subforms Aβ40 and Aβ42, and also levels of ApoE, decline following delipidation treatment when compared to control animals. The results also demonstrate a decline in the ratio of Aβ42 to Aβ40. Taken together, the results indicate that plasma delipidation decreases several parameters in a manner consistent with a reduction in AD pathology.

### EXAMPLE 9

Localization and Quantitation of Amyloid Plaque, Neurofibrillary Tangles and Tau Protein in Cerebral Cortex and Hippocampal Cortex of Aged Rhesus Monkeys Following Reduction in Plasma Lipid Levels

Blood and CSF samples are obtained to measure the baseline parameters described in Example 7 above. Rhesus monkeys are subjected to the plasma delipidation procedure described in Example 7. Other monkeys receive a control procedure in which blood is removed, cells are separated from plasma but the plasma is not treated with solvents to remove lipids. The treated or untreated plasma samples are combined with the blood cells that are separated and then returned to the same monkey from which they are obtained.

Following several delipidation or control treatments, the monkeys in each group are anesthetized, blood and CSF samples are withdrawn for analysis of relevant parameters, and the animals are perfused through the ascending aorta with physiologically buffered saline and fixative for preservation of amyloid plaque, neurofibrillary tangles and tau protein in the cerebral cortex. Such fixatives are known to one of ordinary skill in the art. The calvarium is rapidly removed from each animal, and the brain is dissected free of the dura and cranial nerves. Gross examination is performed. The brain is postfixed and processed using techniques commonly known to histochemists. The brain is blocked into several coronal slices, embedded in paraffin or OCT compound and selected regions of the cerebral cortex (frontal, temporal, parietal and occipital) and hippocampal cortex are sectioned in the coronal plane. The sections are stained for amyloid plaque, neurofibrillary tangles and tau protein using appropriate stains known to one of ordinary skill in the art including but not limited to hematoxylin and eosin, silver stains and Congo red. Positively stained amyloid plaque, neurofibrillary tangles and tau protein is counted in 40 representative sections from each region of the brain.

Results demonstrate that cerebral cortical and hippocampal cortical staining for amyloid plaque, neurofibrillary tangles and tau protein is reduced in animals subjected to plasma delipidation when compared to controls. Gross examination of brains of monkeys receiving plasma delipidation reveals reduced cortical atrophy compared to controls. Taken together, the results indicate that plasma delipidation decreases several parameters associated with AD pathology.

### EXAMPLE 10

Measurement of ApoE, Aβ and Lipid in Plasma and CSF of Humans Before and After Decreasing Lipid Levels in Plasma

Aged human volunteers with high cholesterol levels and demonstrating early signs of AD provide blood samples through venipuncture and CSF samples through lumbar tap. The blood samples are processed to measure the levels of the following parameters in the plasma: LDL, VLDL, HDL, lipids, cholesterol, 24S-cholesterol, triglycerides. The CSF samples are processed to measure the levels of the following parameters: lipid, 24S-cholesterol, ApoE, Aβ and its sub forms including Aβ40, Aβ42 and Aβ43. These molecules are measured using techniques known to one of ordinary skill in the art.

The patients are divided into two groups: one group receives the plasma delipidation procedure. The other group receives a control procedure in which blood is removed, cells are separated from plasma but the plasma is not treated with solvents to remove lipids. The treated or untreated plasma samples are combined with the blood cells that are separated and then returned to the same patient from which they are obtained. The procedure is performed at a sufficient frequency to affect levels of Aβ and ApoE in plasma and CSF. These plasma and CSF parameters establish a baseline for comparison to measurement of these parameters after the delipidation procedure is applied to these patients.

Blood samples and CSF samples are withdrawn from these patients at various intervals during and after the delipidation procedures. Analysis of the plasma and CSF parameters listed above shows that over time, plasma levels of lipid, total cholesterol, of ApoE, and Aβ, and CSF levels of Aβ and ApoE decline in a manner consistent with a reduction in AD pathology.

All patents, publications and abstracts cited above are incorporated herein by reference in their entirety. It should be understood, of course, that the foregoing relates only to preferred embodiments of the present invention and that numerous modifications or alterations may be made therein without departing from the spirit and the scope of the invention as set forth in the appended claims.

What is claimed is:

1. A composition effective to prevent or delay the onset of Alzheimer disease in a patient at risk of developing Alzheimer disease comprising one or more partially delipidated protein particles, one or more partially delipidated lipoprotein particles or a combination thereof.

2. A composition effective to treat Alzheimer disease in a patient with Alzheimer disease comprising one or more partially delipidated protein particles, one or more partially delipidated lipoprotein particles or a combination thereof.

3. The composition of claim 1, wherein the one or more partially delipidated protein particles or the one or more partially delipidated lipoprotein particles are HDL, LDL or VLDL, or a combination thereof.

4. The composition of claim 2, wherein the one or more partially delipidated protein particles or the one or more partially delipidated lipoprotein particles are HDL, LDL or VLDL, or a combination thereof.

5. The composition of claim 1, wherein the one or more partially delipidated protein particles or the one or more partially delipidated lipoprotein particles are prepared by a process comprising:
obtaining blood containing lipid from the patient;
separating cells from the blood to form plasma containing lipid, protein particles and lipoprotein particles;
contacting the plasma containing the lipid and the particles with a first organic solvent capable of extracting lipid;
mixing the fluid and the first organic solvent;
permitting organic and aqueous phases to separate;
collecting the aqueous phase containing reduced lipid content, wherein the aqueous phase contains the one or more partially delipidated protein particles or the one or more partially delipidated lipoprotein particles.

6. The composition of claim 2, wherein the one or more partially delipidated protein or lipoprotein particles are prepared by a process comprising:
obtaining blood containing lipid from the patient;
separating cells from the blood to form plasma containing lipid, protein particles and lipoprotein particles;
contacting the plasma containing the lipid and the particles with a first organic solvent capable of extracting lipid;
mixing the fluid and the first organic solvent;
permitting organic and aqueous phases to separate;
collecting the aqueous phase containing reduced lipid content, wherein the aqueous phase contains the one or more partially delipidated protein particles or the one or more partially delipidated lipoprotein particles.

7. A method for treating Alzheimer disease in a patient diagnosed with Alzheimer disease comprising:
administration of an effective amount of one or more partially delipidated protein particles, one or more partially delipidated lipoprotein particles or a combination thereof, wherein the amount is effective to treat Alzheimer disease in the patient.

8. A method for preventing or delaying the onset of Alzheimer disease in a patient at risk of developing Alzheimer disease comprising:
administration of an effective amount of one or more partially delipidated protein particles, one or more partially delipidated lipoprotein particles or a combination thereof, wherein the amount is effective to prevent or delay the onset of Alzheimer disease in the patient.

9. The method of claim 7, wherein the one or more partially delipidated lipoprotein particles is HDL, LDL or VLDL, or a combination thereof.

10. The method of claim 8, wherein the one or more partially delipidated lipoprotein particles is HDL, LDL or VLDL, or a combination thereof.

11. The method of claim 7, wherein the method reduces amyloid plaque, decreases neurofibrillary tangles, reduces levels of A#, alters a ratio of Aβ40 to Aβ42, affects enzymatic processing of APP, or reduces levels of phosphorylated tau protein.

12. The method of claim 8, wherein the method reduces amyloid plaque, decreases neurofibrillary tangles, reduces levels of Aβ, alters a ratio of Aβ40 to Aβ42, affects enzymatic processing of APP, or reduces levels of phosphorylated tau protein.

13. The method of claim 7, further comprising administration of a therapeutic agent, wherein the therapeutic agent is an agent that affects lipid metabolism or is an agent that affects parameters associated with Alzheimer disease.

14. The method of claim 8, further comprising administration of a therapeutic agent, wherein the therapeutic agent is an agent that affects lipid metabolism or is an agent that affects parameters associated with Alzheimer disease.

15. The method of claim 13, wherein the therapeutic agent is a selected from the group consisting of synthetic HDL compositions, compositions selectively enhancing HDL function with minimal effect on LDL levels, cholesteryl ester transfer protein inhibitors, cholesterol level lowering agents and triglyceride level lowering agents in a pharmaceutically acceptable vehicle, and combinations thereof.

16. The method of claim 14, wherein the therapeutic agent is a selected from the group consisting of synthetic HDL compositions, compositions selectively enhancing HDL function with minimal effect on LDL levels, cholesteryl ester transfer protein inhibitors, cholesterol level lowering agents and triglyceride level lowering agents in a pharmaceutically acceptable vehicle, and combinations thereof.
